(19) United States
(12) Patent Application Publication
(10) Pub. No.: US 2025/0251478 A1
(43) Pub. Date: Aug. 7, 2025

(54) DEEP LEARNING BASED DENOISING OF MR IMAGES

(71) Applicant: KONINKLIJKE PHILIPS N.V., EINDHOVEN (NL)

(72) Inventors: Nils Thorben Gessert, Hamburg (DE); Christian Wuelker, Hamburg (DE); Tim Nielsen, Hamburg (DE)

(21) Appl. No.: 18/851,098

(22) PCT Filed: Mar. 21, 2023
(86) PCT No.: PCT/EP2023/057109

§ 371 Date: Sep. 26, 2024

(30) Foreign Application Priority Data
Mar. 31, 2022 (NL) 2031467

(51) Int. Cl. G01R 33/56; G06T 5/60; G06T 5/70

(52) U.S. Cl. CPC G01R 33/5608; G06T 5/60; G06T 5/70; G06T 2207/10088; G06T 2207/20081; G06T 2207/20084; G06T 2207/20092; G06T 2207/30004

(57) ABSTRACT

The invention relates to a method of MR imaging of an object positioned in the examination volume of an MR system (1). It is an object of the invention to provide a deep learning-based denoising approach that overcomes the Rician bias problem. As a solution, the invention proposes a method comprising the following steps: a) subjecting the object to an imaging sequence comprising RF pulses and switched magnetic field gradients, whereby MR signals are generated, b) acquiring the MR signals, c) reconstructing a complex-valued MR image from the acquired MR signals, d) denoising the MR image using a deep learning algorithm that operates on the real and the imaginary parts of the MR image, and c) computing a magnitude MR image from the denoised complex-valued MR image. According to an aspect of the invention, the deep learning algorithm uses a set of denoising models that are trained using different loss functions. In this way, a trade-off between noise removal and preservation of small image details can be controlled. Moreover, the invention relates to an MR system (1) and to a computer program for an MR system (1).

### FIELD OF THE INVENTION

The invention relates to the field of magnetic resonance (MR) imaging. It concerns a method of MR imaging of an object placed in the examination volume of an MR system. The invention also relates to an MR system and to a computer program to be run on an MR system.

### BACKGROUND OF THE INVENTION

Image-forming MR methods which utilize the interaction between magnetic fields and nuclear spins in order to form two-dimensional or three-dimensional images are widely used nowadays, notably in the field of medical diagnostics, because for the imaging of soft tissue they are superior to other imaging methods in many respects, do not require ionizing radiation and are usually not invasive.

MR image quality improvement is crucial for diagnostic confidence and quality. A low signal to noise ratio (SNR) can substantially degrade the quality of MR images. One way to improve SNR is the use of multiple acquisitions at the same locations in k-space and averaging the resulting signal data. Naturally, this process is time-consuming and expensive, which creates a need for other solutions. Alternatively, denoising methods can be used to improve the SNR of acquired noisy MR images. Besides conventional filtering techniques, artificial intelligence (AI)/deep learning-based methods have recently gained popularity for MR image denoising (see Jiang, D., Dou, W., Vosters, L., Xu, X., Sun, Y., Tan, T., 2018, “Denoising of 3D magnetic resonance images with multi-channel residual learning of convolutional neural network”, Japanese Journal of Radiology, 36, vol. 9, p. 566-574).

Noise of fully-sampled MR imaging scans is typically modeled as Gaussian noise in the real and imaginary parts of the complexed-valued MR image. However, MR images are typically displayed as magnitude images, leading to transformation of the original Gaussian noise. The resulting intensity distribution in magnitude MR images follows a Rice (Rician) distribution which is intensity-dependent. For high-signal intensity regions, this distribution is similar to a Gaussian distribution. However, for low-signal intensity image regions the distribution is close to a Rayleigh distribution with non-zero mean. For low-SNR images this leads to the so-called Rician noise bias (see Cárdenas-Blanco, A., Tejos, C., Irarrazaval, P., Cameron, I., 2008, “Noise in magnitude magnetic resonance images”, Concepts in Magnetic Resonance Part A: An Educational Journal, 32, vol. 6, p. 409-416).

The Rician noise bias is a crucial problem for the known deep learning-based denoising methods since the Rician noise bias cannot be completely removed. As a result, a denoised but biased MR image is produced.

The US-patent application US2022/026516 concerns a method for correcting phase and reducing noise in an magnetic resonance phase image. Phase information may be corrupted due to eddy current effects, physiological noise or gradient imperfections in the spatial encoding. A deep neural network is employed to denoise the phase images. Denoising of complex images is done by multiplying the magnitude image by the phase corrected pristine images.

### SUMMARY OF THE INVENTION

From the foregoing it is readily appreciated that there is a need for an improved method of MR image denoising. It is thus an object of the invention to provide a denoising approach that overcomes the Rician bias problem. In this text, the expression denoising is defined as noise reduction, which noise reduction does not necessarily result in removing all noise. It is thus possible to discuss a degree of noise reduction achieved by the denoising.

In accordance with the invention, a method of MR imaging of an object positioned in the examination volume of an MR system is disclosed. The method comprises the steps of:
a) subjecting the object to an imaging sequence comprising RF pulses and switched magnetic field gradients, whereby MR signals are generated,
b) acquiring the MR signals.
c) reconstructing a complex-valued MR image from the acquired MR signals,
d) denoising the MR image using a deep learning algorithm that operates on the real and the imaginary parts of the MR image, and
e) computing a magnitude MR image from the denoised complex-valued MR image.

According to the invention, an MR signal data set is acquired in a conventional fashion. The MR signals are acquired in k-space using quadrature detection delivering a real and an imaginary signal part. A conventional image reconstruction technique, such as, e.g., Fourier transformation, produces a complex-valued MR image indicating the phase and the amplitude of the MR signal for each image position. The invention proposes to overcome the Rician bias problem by performing a complex denoising using a deep learning algorithm. The deep learning algorithm operates on the real and imaginary parts of the MR image instead as on the magnitude MR image (as it is the conventional approach). In this way, the invention exploits that the noise characteristic of the individual real and imaginary parts is Gaussian which is significantly easier to remove. Potential noise is removed from the real and imaginary part of the image before it is finally converted into a magnitude MR image for display to a user. As a result, a Rician noise bias is avoided before it comes into existence.

The proposed complex denoising method processes the real and imaginary parts of the MR image. Thus, the denoising procedure should be placed inside the MR reconstruction pipeline (where the real and imaginary parts are available) before the final magnitude MR image is produced. However, the technique proposed by the invention can also be used independently. e.g. as a mere post-processing step independent of the actual MR signal acquisition, and also for other types of imaging modalities besides MR imaging, where complex images are processed. Hence, the disclosure of the invention also encompasses a computer-implemented method for denoising an image, comprising the steps:
providing a complex-valued image.
denoising the image using a deep learning algorithm that operates on the real and the imaginary parts of the image, and
computing a magnitude image from the denoised complex-valued image.

In an embodiment, each of the real and the imaginary parts is split into a high-frequency part and a low-frequency part prior to the denoising of the MR image. The terms high-frequency and low-frequency refer to spatial frequencies. i.e. to image information associated with peripheral and central portions of k-space respectively. To obtain better generalizability across different MR imaging contrasts, the complex denoising should operate only on the high-frequency part of the real part and the high-frequency part of the imaginary part of the MR image. In this way, the low-frequency contrast-specific image information is preserved and the denoising only deals with the high-frequency noise and structure in the MR image. The deep learning algorithm produces a denoised version of the high-frequency real and imaginary part respectively. After the denoising step, the denoised high-frequency real part and the low-frequency real part are combined into a final real part and also the denoised high-frequency imaginary part and the low-frequency imaginary part are combined into a final imaginary part. In other words, the low-frequency parts are each added back to the denoised high-frequency real and imaginary parts. The magnitude MR image is computed from the final real part and the final imaginary part. The magnitude MR image is then displayed to the user and/or stored to Dicom. Further image processing steps may be applied before the magnitude MR image is shown to the user.

As mentioned above, the Rician noise bias is a crucial problem in known deep learning-based denoising methods. In particular, if the deep learning model operates only on the high-frequency part of the MR image, it is not able to make changes to the low-frequency part of the MR image. For magnitude MR images with Rician noise bias, this means that the bias in the low-frequency image part cannot be removed. As a result, a denoised but biased image is produced. In contrast to this, the method of the invention can apply a noise reduction that operates only on the high-frequency part without resulting in any bias in the final MR image. This is because the method of the invention, in which the deep learning algorithm processes the complex image data, is bias-free from the outset.

In an embodiment of the invention, the deep learning algorithm may use a complex-valued convolutional neural network. In this way, the neural network is enabled to operate on the real and imaginary parts of the MR image. This means that the neural network performs complex convolutions for processing of the real and imaginary parts of the MR image.

A further issue with deep learning-based MR image denoising methods is that they often suffer from over-smoothing. For example, a high-performing deep learning model that is able to remove almost all noise in an MR image usually produces an image with an unnatural look. Furthermore, the strong smoothing can eliminate small anatomical features that might be important for diagnosis. Thus, while noise removal is helpful for improving image quality, too much noise removal can also be detrimental. For these reasons, the invention proposes that the deep learning algorithm uses a set of denoising models that are trained using different loss functions. Different loss functions within the meaning of the invention can be differently parametrized versions of the same specialized loss function in which the denoising strength can be set to a desired level. In a possible embodiment, this is achieved by loss functions that differ by their respective weightings of variance and bias, wherein bias is a measure of the denoising model's capability to retain image details, and variance is a measure for the degree of noise reduction achieved by the denoising model. In this way, a trade-off between noise reduction and preservation of small image details can be precisely controlled.

The proposed variable weighting of variance and bias can be implemented in combination with the above-described denoising of the real and the imaginary parts of the MR image, or independently thereof. e.g. for conventional deep learning-based denoising operating directly on a magnitude image. To this end, the disclosure of the invention encompasses a computer-implemented method for denoising an image, comprising the steps:
providing an image,
providing a set of deep learning-based denoising models that are trained using different loss functions, wherein the loss functions differ by their respective weightings of variance and bias, wherein bias is a measure of the denoising model's capability to retain image details, and variance is a measure for the degree of noise reduction achieved by the denoising model.
denoising the image using a denoising model which is selected interactively by a user from said set. The user can try different denoising models and, after reviewing the respective denoised images, select the model that he believes represents the best compromise between noise reduction and detail accuracy. The proposed denoising method can be applied to any type of imaging modality, in particular MR. CT. PET. In particular, low-cost MR systems that are affected by noise more severely will benefit from the method of the invention as denoising and tuning of the denoising strength are of high practical relevance in these systems.

The method of the invention described thus far can be carried out by means of an MR system including at least one main magnet coil for generating a uniform, steady magnetic field B₀ within an examination volume, a number of gradient coils for generating switched magnetic field gradients in different spatial directions within the examination volume, at least one body RF coil for generating RF pulses within the examination volume and/or for receiving MR signals from a body of a patient positioned in the examination volume, a control unit for controlling the temporal succession of RF pulses and switched magnetic field gradients, and a reconstruction unit for reconstructing MR images from the received MR signals. The method of the invention can be implemented by a corresponding programming of the reconstruction unit and/or the control unit of the MR system.

The method of the invention can be advantageously carried out on most MR systems in clinical use at present. To this end it is merely necessary to utilize a computer program by which the MR system is controlled such that it performs the above-explained method steps of the invention. The computer program may be present either on a data carrier or be present in a data network so as to be downloaded for installation in the control unit of the MR system.

### BRIEF DESCRIPTION OF THE DRAWINGS

The enclosed drawings disclose preferred embodiments of the present invention. It should be understood, however, that the drawings are designed for the purpose of illustration only and not as a definition of the limits of the invention. In the drawings:

FIG. 1 shows an MR system for carrying out the method of the invention:

FIG. 2 illustrates the method of the invention as a flow diagram:

FIG. 3 illustrates the method of the invention compared to conventional deep learning-based image denoising:

FIG. 4 illustrates a further aspect of the invention as a flow chart.

### DETAILED DESCRIPTION OF THE EMBODIMENTS

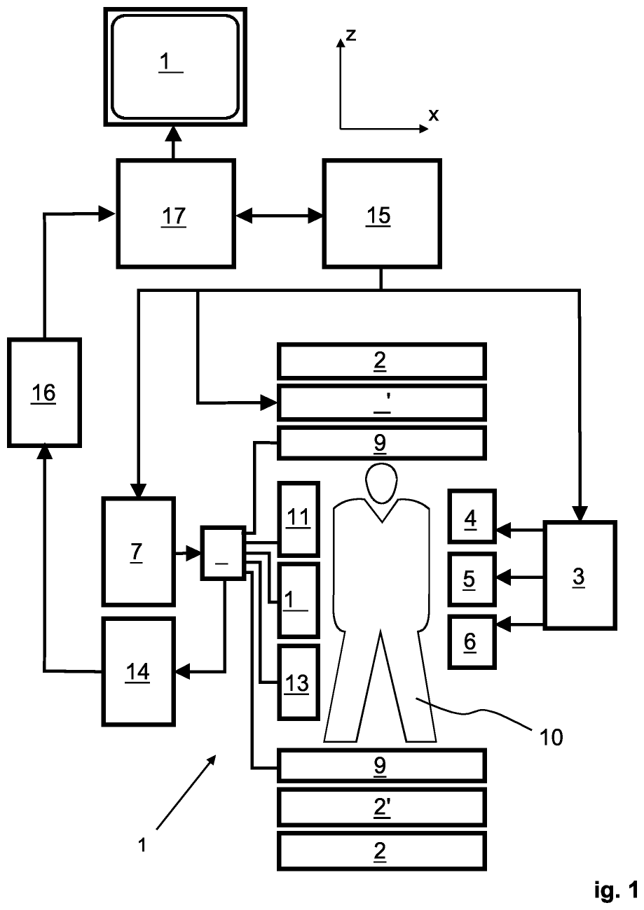

With reference to FIG. 1, an MR system 1 is shown as a block diagram. The device comprises superconducting or resistive main magnet coils 2 such that a substantially uniform, temporally constant main magnetic field B₀ is created along a z-axis through an examination volume. The device further comprises a set of (1^{st}, 2^{nd}, and—where applicable—3^{rd} order) shimming coils 2′, wherein the current flow through the individual shimming coils of the set 2′ is controllable for the purpose of minimizing B₀ deviations within the examination volume.

A magnetic resonance generation and manipulation system applies a series of RF pulses and switched magnetic field gradients to invert or excite nuclear magnetic spins, induce magnetic resonance, refocus magnetic resonance, manipulate magnetic resonance, spatially and otherwise encode the magnetic resonance, saturate spins, and the like to perform MR imaging.

More specifically, a gradient amplifier 3 applies current pulses or waveforms to selected ones of whole-body gradient coils 4, 5 and 6 along x, y and z-axes of the examination volume. A digital RF frequency transmitter 7 transmits RF pulses or pulse packets, via a send/receive switch 8, to a body RF coil 9 to transmit RF pulses into the examination volume. A typical MR imaging sequence is composed of a packet of RF pulse segments of short duration which, together with any applied magnetic field gradients, achieve a selected manipulation of nuclear magnetic resonance signals. The RF pulses are used to saturate resonance, excite resonance, invert magnetization, refocus resonance, or manipulate resonance and select a portion of a body 10 positioned in the examination volume. The MR signals are also picked up by the body RF coil 9.

For generation of MR images of limited regions of the body 10 or for scan acceleration by means of parallel imaging, a set of local array RF coils 11, 12, 13 are placed contiguous to the region selected for imaging. The array coils 11, 12, 13 can be used to receive MR signals induced by body coil RF transmissions.

The resultant MR signals are picked up by the body RF coil 9 and/or by the array RF coils 11, 12, 13 and demodulated by a receiver 14 preferably including a pre-amplifier (not shown). The receiver 14 is connected to the RF coils 9, 11, 12 and 13 via the send/receive switch 8.

A host computer 15 controls the shimming coils 2′ as well as the gradient pulse amplifier 3 and the transmitter 7 to generate any of a plurality of MR imaging sequences, such as echo planar imaging (EPI), echo volume imaging, gradient and spin echo imaging, fast spin echo imaging, and the like. For the selected sequence, the receiver 14 receives a single or a plurality of MR signals in rapid succession following each RF excitation pulse, wherein the MR signals are received in quadrature such that the MR signals are complex-valued and comprise a real part and an imaginary part. A data acquisition system 16 performs analog-to-digital conversion of the received signals and converts the MR data samples to a digital format suitable for further processing. In modern MR systems the data acquisition system 16 is a separate computer which is specialized in acquisition of raw image data.

Ultimately, the digital raw image data are reconstructed into an image representation by a reconstruction processor 17 which applies a Fourier transform or other appropriate reconstruction algorithms. The MR image may represent a planar slice through the patient, an array of parallel planar slices, a three-dimensional volume, or the like. The image is then stored in an image memory where it may be accessed for converting slices, projections, or other portions of the image representation into appropriate format for visualization, for example via a video monitor 18 which provides a man-readable display of the resultant MR image.

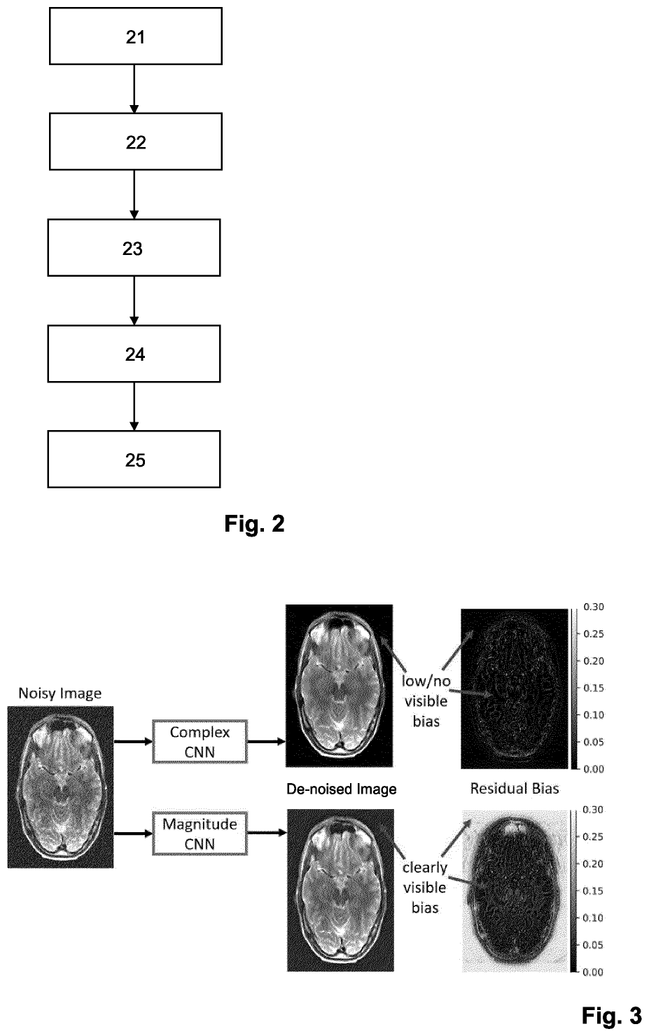

The reconstruction processor 17 is programmed to execute the method of the invention described herein above and in the following with further reference to FIGS. 2-4.

In an embodiment of the invention illustrated in FIG. 2, an imaging sequence is applied in step 21 which imaging sequence comprises RF excitation pulses and switched magnetic field gradients in the read-out and phase-encoding directions x and y and in the slice-selection direction z. A MR signal data set is acquired in multiple repetitions of the sequence using different gradient waveforms in the x-/y-directions and/or in the z-direction in order to completely cover the required region of k-space by a suitable (e.g. radial or Cartesian) k-space sampling pattern. Step 21 also encompasses the reconstruction of a complex-valued MR image from the acquired MR signals. e.g. by Fourier transformation as described above.

In step 22, a frequency split of the reconstructed MR image is performed. The real and the imaginary part of the MR image each are split into a low-frequency and a high-frequency part. This can be implemented, e.g., by filtering (convolution) with a Gaussian kernel of a suitably selected width that produces the low-frequency parts. The high-frequency parts are obtained by subtracting the respective low-frequency parts from the real and imaginary parts of the original MR image.

In step 23, a denoising of the MR image is performed using a deep learning algorithm that operates on the high-frequency real and the imaginary parts of the MR image.

Three different versions of the complex denoising step can be considered that are all based on convolutional neural networks (CNNs).

First, a basic channel-stacking approach is conceivable, in which the high-frequency real and imaginary parts are stacked along the first CNN layer's channel dimension, similar to the color channel dimension in CNNs used for processing of natural images. Unlike conventional denoising CNNs, the model outputs two denoised images, each corresponding to the real and imaginary parts of the complex MR image at the input of the CNN.

As a second option, a complex-valued CNN can be conceived for processing of the high-frequency real and imaginary parts of the complex MR image. In this case, convolutions can be performed similar to a multiplication of complex numbers. In each convolutional layer, the feature maps can be computed by

$${\hat{x}}_{R} + j {\hat{x}}_{I} = ( {x}_{R} * {w}_{R} - {x}_{I} * {w}_{I} ) + j ( {x}_{R} * {w}_{I} + {x}_{I} * {w}_{R} )$$

where x is a complex-valued feature map and w a complex-valued convolutional kernel.

As a further alternative an independent processing scheme can be conceived in which the high-frequency real and imaginary parts are processed (e.g. in a sequential order) by the same CNN.

All CNN models can be trained in a residual mode. This means, that the CNN receives a noisy training image as input and predicts the noise that is present in the image. The noise is then subtracted from the input image and compared to a noise-free ground-truth for calculation of the used training loss function. The CNN is then trained using a gradient descent optimization algorithm wherein the weights in the CNN are updated using a backpropagation of error technique. The gradient descent refers to a gradient of the training loss function which the training algorithm seeks to minimize by changing the weights of the CNN, meaning that the optimization algorithm is navigating down (descending) the gradient of the loss function error. Noisy training images for this procedure can be generated by adding Gaussian noise to the real and imaginary parts of existing MR images. In the last variant mentioned above (independent processing of the high-frequency real and imaginary parts by the same CNN) the CNN can also be trained with natural (i.e. non-MR) images such that no access to high-quality MR images is required. This is possible because the noise in the real and imaginary parts of the MR image can be considered uncorrelated.

Processing step 24 is the re-combination into a denoised final MR image. The denoised high-frequency real and imaginary parts are added to their respective low-frequency counterparts.

Then, in step 25, the final MR image is obtained by taking the magnitude of the denoised complex-valued MR image.

In an embodiment of the invention, the described denoising method can advantageously be implemented in the MR system's I reconstruction processor 17 as the real and imaginary parts of the MR image need to be available: First, the frequency split described in step 22 is applied, resulting in a low-frequency and high-frequency part each for the real and imaginary part of the MR image. Then, the high-frequency part of the image is processed by the complex deep learning model described in step 23. The output of the model is the noise that is present in the high-frequency part of the real and imaginary parts of the MR image. Then, the predicted noise is subtracted from the original high-frequency parts. If desired, the user can control the amount of noise that is removed interactively. Finally, the combination described in step 24 is applied and the final denoised MR magnitude image is computed in step 25. The magnitude MR image is displayed to a user and can be stored to Dicom.

FIG. 3 shows the results of an exemplary implementation of the method of the invention in comparison to conventional deep learning-based magnitude denoising. A noisy magnitude MR image is shown on the left. In the upper row, the denoising result of the invention (complex CNN) is shown. In the lower row, a conventional magnitude CNN denoising is shown. While the magnitude CNN denoising leads to visible bias inside and outside the anatomy, the complex CNN does not show any visible bias. The residual bias maps shown on the right refer to the absolute difference between the denoised image and a corresponding noise-free ground-truth image.

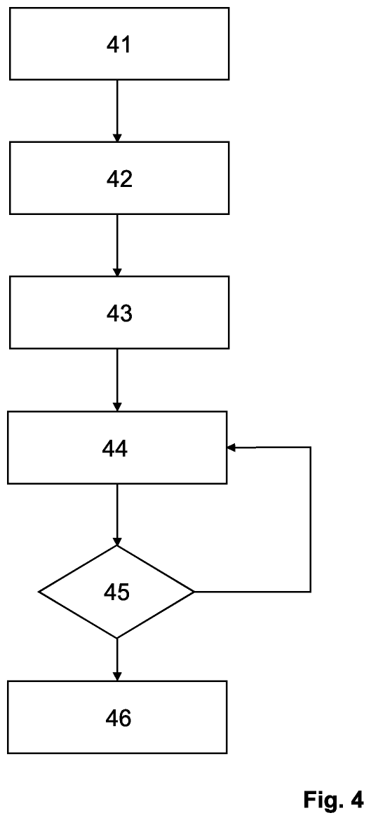

FIG. 4 illustrates a further aspect of the invention which provides for controlling the denoising strength of a deep learning-based denoising method. A noise simulation is performed in step 41 which is able to produce realistic noise scenarios from noise-free ground-truth images. For a Rician noise scenario (e.g. in SENSE scans). Gaussian noise can be added to the real and imaginary parts of the MR image. For a colored noise scenario (e.g. C-SENSE), noise-free ground-truth images can be undersampled in k-space to produce noise realizations. In this way, a set of training MR images is generated.

In step 42, a set of deep learning denoising models is trained using the generated training MR images. Each model receives the noisy training images as its input and predicts the corresponding noise-free ground-truth images at its output. Alternatively, the model can be trained to predict the noise that is present in the images. This predicted noise is then subtracted from the input, also leading to a denoised image in each case. The model can have any architecture, a typical choice would again be a CNN. Each denoising model of the set is associated with a different loss function that is used for training of the model. The loss functions differ by their respective weightings of variance and bias, wherein bias is a measure of the denoising model's capability to retain image details, and variance is a measure for the degree of noise reduction achieved by the denoising model. Bias can be expressed as the error between the mean model output across multiple processed noisy training images and respective noise-free ground-truth images. For additive Gaussian noise, a noisy training image can be defined as x=x+n where x is a noise-free image and n is the added noise with n˜N (0,σ). Correspondingly, the variance can be expressed as the pixel-wise standard deviation across multiple processed noisy training images. Thus, the loss function that is to be optimized (minimized) in the training step 42 can be expressed as

$${ℒ}_{BV} = ( 1 - α ) {ℒ}_{Bias} + {αℒ}_{Var}$$

$$with$$

$${ℒ}_{Bias} = {ℒ}_{1} ( \frac{1}{M} {\sum }_{i = 1}^{M} {\hat{x}}_{i} , x )$$

$$and$$

$${ℒ}_{Var} = \frac{1}{D} {\sum }_{j = 1}^{D} σ ( \hat{x} )$$

where [symbol] ₁ is the L1 norm (optionally the L2 norm or any other distance measure can be used as well), xᵢ is a single denoised training image and x is a tensor containing all M denoised training images. D is the total number of pixels or voxels in the image. σ is a function that calculates the pixel-wise standard-deviation across the different training images. As an alternative, this function can also calculate the variance. The parameter a is tunable and allows for precise control of the trade-off between retaining detail and denoising strength. The set of denoising models is thus trained for different values of α. A different value of a is associated with each denoising model of the set.

In step 43, an MR image to be denoised is provided. The MR image can be a complex MR image acquired as described above or a magnitude MR image.

In step 44, the MR image is denoised using one of the previously trained denoising models for a selected value of a and the denoised image is displayed to a user.

In step 45, the user assesses the displayed image and decides whether the result of the denoising procedure is satisfactory. If this is not the case, step 44 is repeated for a different value of α.

If the result is deemed satisfactory, the procedure ends in step 46. The final denoised MR image can be stored to Dicom for further use in diagnosis.

Various aspects and embodiments of the invention are summarized in the following clauses:

Clause 1. Method of MR imaging of an object (10) positioned in the examination volume of an MR system (1), the method comprising the steps of:
a) subjecting the object (10) to an imaging sequence comprising RF pulses and switched magnetic field gradients, whereby MR signals are generated,
b) acquiring the MR signals,
c) reconstructing a complex-valued MR image from the acquired MR signals,
d) denoising the MR image using a deep learning algorithm that operates on the real and the imaginary parts of the MR image, and
e) computing a magnitude MR image from the denoised complex-valued MR image.

Clause 2. Method of Clause 1, wherein each of the real and the imaginary part is split into a high-frequency part and a low-frequency part prior to the denoising of the MR image.

Clause 3. Method of Clause 2, wherein the denoising operates only on the high-frequency part of the real part and the high-frequency part of the imaginary part of the MR image.

Clause 4. Method of Clause 3, wherein the denoised high-frequency real part and the low-frequency real part are combined into a final real part and also the denoised high-frequency imaginary part and the low-frequency imaginary part are combined into a final imaginary part, wherein the magnitude MR image is computed from the final real part and the final imaginary part.

Clause 5. Method of any one of Clauses 1-4, wherein the deep learning algorithm uses a convolutional neural network.

Clause 6. Method of Clause 5, wherein the convolutional neural network is complex-valued.

Clause 7. Method of any one of Clauses 1-6, wherein the deep learning algorithm uses a set of denoising models that are trained using different loss functions.

Clause 8. Method of Clause 7, wherein the loss functions differ by their respective weightings of variance and bias, wherein bias is a measure of the denoising model's capability to retain image details, and variance is a measure for the degree of noise reduction achieved by the denoising model.

Clause 9. Method of Clause 7 or 8, wherein one of the denoising models from said set is selected interactively by a user for denoising the MR image.

Clause 10. Computer-implemented method for denoising an image, the method comprising the steps:
providing a complex-valued image,
denoising the image using a deep learning algorithm that operates on the real and the imaginary parts of the image, and
computing a magnitude image from the denoised complex-valued image.

Clause 11. Computer-implemented method for denoising an image, the method comprising the steps:
providing an image,
providing a set of deep learning-based denoising models that are trained using different loss functions, wherein the loss functions differ by their respective weightings of variance and bias, wherein bias is a measure of the denoising model's capability to retain image details, and variance is a measure for the degree of noise reduction achieved by the denoising model,
denoising the image using a denoising model which is selected interactively by a user from said set.

Clause 12. MR system including at least one main magnet coil (2) for generating a uniform, steady magnetic field B₀ within an examination volume, a number of gradient coils (4, 5, 6) for generating switched magnetic field gradients in different spatial directions within the examination volume, at least one RF coil (9) for generating RF pulses within the examination volume and/or for receiving MR signals from an object (10) positioned in the examination volume, a control unit (15) for controlling the temporal succession of RF pulses and switched magnetic field gradients, and a reconstruction unit (17) for reconstructing MR images from the received MR signals, wherein the MR system (1) is arranged to perform the method of any one of Clauses 1-11.

Clause 13. Computer program comprising instructions which, when the program is executed by a computer, preferably by a reconstruction unit (17) of an MR system (1), cause the computer to carry out the method of any one of Clauses 1-11.

1. Method A method of magnetic resonance (MR) imaging of an object positioned in the examination volume of an MR system, the method comprising the steps of:
a) subjecting the object to an imaging sequence comprising RF pulses and switched magnetic field gradients, whereby MR signals are generated,
b) acquiring the MR signals,
c) reconstructing a complex-valued MR image from the acquired MR signals, d) denoising the MR image using a deep learning algorithm that operates on the real and the imaginary parts of the MR image, and
e) computing a magnitude MR image from the denoised complex-valued MR image.

2. The method of claim 1, wherein each of the real and the imaginary part is split into a high-frequency part and a low-frequency part prior to the denoising of the MR image.

3. The method of claim 2, wherein the denoising operates only on the high-frequency part of the real part and the high-frequency part of the imaginary part of the MR image.

4. The method of claim 3, wherein the denoised high-frequency real part and the low-frequency real part are combined into a final real part and also the denoised high-frequency imaginary part and the low-frequency imaginary part are combined into a final imaginary part, wherein the magnitude MR image is computed from the final real part and the final imaginary part.

5. The method of claim 1, wherein the deep learning algorithm uses a convolutional neural network.

6. The method of claim 5, wherein the convolutional neural network is complex-valued.

7. The method of claim 1, wherein the deep learning algorithm uses a set of denoising models that are trained using different loss functions.

8. The method of claim 7, wherein the loss functions differ by their respective weightings of variance and bias, wherein bias is a measure of the denoising model's capability to retain image details, and variance is a measure for the degree of noise reduction achieved by the denoising model.

9. The method of claim 7, wherein one of the denoising models from said set is selected interactively by a user for denoising the MR image.

10. A computer-implemented method for denoising an image, the method comprising:
providing a complex-valued image,
denoising the image using a deep learning algorithm that operates on the real and the imaginary parts of the image, and
computing a magnitude image from the denoised complex-valued image.

11. The computer-implemented method for denoising an image, the method comprising the steps:
providing an image,
providing a set of deep learning-based denoising models that are trained using different loss functions, wherein the loss functions differ by their respective weightings of variance and bias, wherein bias is a measure of the denoising model's capability to retain image details, and variance is a measure for the degree of noise reduction achieved by the denoising model,
denoising the image using a denoising model which is selected interactively by a user from said set.

12. A magnetic resonance (MR) system including at least one main magnet coil for generating a uniform, steady magnetic field (B₀) within an examination volume, a number of gradient coils for generating switched magnetic field gradients in different spatial directions within the examination volume, at least one RF coil for generating RF pulses within the examination volume and/or for receiving MR signals from an object positioned in the examination volume, a control unit for controlling the temporal succession of RF pulses and switched magnetic field gradients, and a reconstruction unit for reconstructing MR images from the received MR signals, wherein the MR system is arranged to perform the method of claim 1.

13. A computer program comprising instructions stored on a non-transitory computer readable medium which, when the program is executed by a computer, of an MR system, cause the computer to carry out the method of claim 1.
